(19) United States
(12) Patent Application Publication
(10) Pub. No.: US 2024/0342073 A1
(43) Pub. Date: Oct. 17, 2024

(54) AGENT FOR DYEING KERATIN MATERIAL, IN PARTICULAR HUMAN HAIR, CONTAINING AMINOSILICONES AND PLATELET-TYPE METALLIC PIGMENTS

(71) Applicant: HENKEL AG & CO. KGAA, Düsseldorf (DE)

(72) Inventors: Constanze KRUCK, Grevenbroich (DE); Gabriele WESER, Essen (DE); Sandra HILBIG, Bochum (DE); Melanie MOCH, Dormagen (DE)

(21) Appl. No.: 18/294,992

(22) PCT Filed: Jun. 20, 2022
(86) PCT No.: PCT/EP2022/066692

§ 371 Date: Feb. 2, 2024

(30) Foreign Application Priority Data
Aug. 4, 2021 (DE) 10 2021 208 460.5

(51) Int. Cl. A61K 8/898; A61K 8/26; A61Q 5/06

(52) U.S. Cl. CPC A61K 8/898; A61K 8/26; A61Q 5/065; A61K 2800/412; A61K 2800/43; A61K 2800/805

(57) ABSTRACT

An agent for dyeing keratin material, such as human hair, is described. The agent includes at least one amino-functionalized silicone polymer, and at least one platelet-type metallic pigment.

The present application relates to an agent for dyeing keratin material, in particular human hair, which contains at least one amino-functionalized silicone polymer and at least one platelet-type metallic pigment.

A further subject matter of this application is a method for dyeing keratin material, in particular human hair, the agent described above being applied to the keratin material and, if appropriate, is rinsed out again after an exposure time of 30 seconds to 45 minutes.

Changing the shape and color of keratin material, in particular human hair, represents an important field of modern cosmetics. To change the hair color, the skilled artisan is familiar with a variety of dyeing systems depending on the dyeing requirements. Oxidation dyes are typically used for permanent, intense dyeing with good fastness properties and good gray coverage. Such dyeing agents contain oxidation dye precursors, what are known as developer components and coupler components, which together form the actual dyes under the influence of oxidizing agents such as, for example, hydrogen peroxide. Oxidation dyes are characterized by very long-lasting color results.

When using direct dyes, dyes which are already formed diffuse out of the dyeing agent into the hair fiber. In comparison with oxidative hair dyeing, the colors obtained with direct dyes have a lower durability and a more rapid washing out. Colors with direct dyes usually remain on the hair for a period of between 5 and 20 hair washes.

The use of color pigments for brief changes in color on the hair and/or the skin is known. Color pigments are generally understood to mean insoluble dyeing substances. These are present undissolved in the form of small particles in the dyeing formulation and are only deposited from the outside onto the hair fibers and/or the skin surface. They can therefore generally be removed again without leaving residue by washing a few times with surfactant-containing cleaning agents. Various products of this type by the name of hair mascara are available on the market.

If the user desires particularly long-lasting dyeing, the use of oxidative dyeing agents has hitherto been the only option. However, despite multiple optimization attempts, an unpleasant ammonia odor or amine odor cannot be completely avoided in oxidative hair dyeing. The hair damage that remains associated with the use of the oxidative dyeing agents also has a disadvantageous effect on the hair of the user. A continuing challenge is therefore the search for alternative, high-performance dyes and dyeing processes. Recently, there has been a particular focus on dyeing systems based on pigments.

In pigment-based dyeing agents, the major challenge is to bind the pigments to the keratin material uniformly and for as long as possible. Because the pigments cannot diffuse into the keratin material, they are generally fixed to the surface of the keratin material via various adhesive materials. Suitable fixing materials are, for example, polymer compounds such as aminosilicones which form a layer or a film on the keratin material into which the pigments are then embedded. Depending on the strength or hydrophobicity of the dyed layer, it can remain on the keratin material over the duration of several hair washes and in this way produce dyes with improved wash fastness.

When used on hair, a major disadvantage that may be associated with the formation of the dyed films is the impairment of the feel of the hair. In particular when using pigments or aminosilicones in larger quantity ranges, the hair can feel heavy, greasy, unpleasant, sticky or even rough. There is therefore a great need for improvement with regard to finding pigment-based dyeing agents which are both long-lasting and cover well and also do not adversely affect the feel of the hair.

It was the object of the present invention to provide a dyeing agent which makes it possible to fix pigments to the hair in an extremely durable manner. When using the agent in a dyeing method, particularly intense dyeing results should be achieved with outstanding covering power with good wash fastness. At the same time, the hair should also have a pleasant, smooth and non-heavy hair feel after the dyeing.

Surprisingly, it has been found that this aforementioned object can be achieved in an outstanding manner when keratin material, in particular human hair, is dyed with an agent which contains at least one amino-functionalized silicone polymer and at least one platelet-type metallic pigment.

A first subject matter of the present invention is an agent for dyeing keratin material, in particular human hair, containing:
(a1) at least one amino-functionalized silicone polymer, and
(a2) at least one platelet-type metallic pigment.

Within the context of the work leading to this invention, it was possible to discover that especially good color results were obtained when the platelet-type metallic pigment(s), in admixture with at least one aminosilicone, were applied to the keratin material, in particular human hair. The dyeings obtained in this way are distinguished by a particularly high covering power and a uniform color result. If the platelet-type metallic pigment has been selected from a correspondingly bright metal, a brightening effect could also be produced optically, so that the hair dyed with the pigment appeared brighter compared to the original hair color. It has been found to be very particularly surprising in this context that the hair still feels natural, smooth and unheavy even after higher concentrations of aminosilicone (a1) and platelet-type metallic pigment (a2) are used. In this way, it was possible to achieve particularly uniform dyeings with high covering power while obtaining the natural hair feel.

### Keratin Material

Keratin material is understood to mean hair, skin, and nails (such as, for example, fingernails and/or toenails). Furthermore, wool, furs and feathers also fall under the definition of the keratin material.

Keratin material is preferably understood to be human hair, human skin and human nails, in particular fingernails and toenails. Keratin material is very particularly preferably understood to mean human hair.

### Dyeing Agent

Within the scope of this invention, the term “dyeing agent” is used for a coloring of the keratin material, in particular hair, brought about by use of pigments. With this coloring, the pigments as coloring compounds are deposited in a particularly homogeneous and uniform film on the surface of the keratin material.

According to the invention, the dyeing agent represents a ready-to-use agent. This ready-to-use agent can, for example, be filled into a container and applied to the keratin material in this form without further dilution, mixing or other method steps. For reasons of storage stability, however, it has been found to be very particularly preferred for the ready-to-use cosmetic agent to be prepared by the hairdresser or user only shortly before use. To produce the ready-to-use agent, it is possible, for example, to mix the amino-functionalized silicone polymer(s) (a1) with the platelet-type metallic pigment(s) (a2), the constituents (a1) and/or (a2) either being provided in the form of a concentrate or it also being possible for them to be present separately in suitable separate cosmetic carriers as an emulsion/dispersion. The agents can be mixed, for example, by stirring or shaking.

In other words, a first subject matter of the present invention is an agent for dyeing keratin material, in particular human hair, which agent is preferably ready to use and contains in a cosmetic carrier:
(a1) at least one amino-functionalized silicone polymer, and
(a2) at least one platelet-type metallic pigment.

### Amino-Functionalized Silicone Polymers (a1)

As a first ingredient essential to the invention, the agent according to the invention contains at least one amino-functionalized silicone polymer (a1). The amino-functionalized silicone polymer can alternatively also be referred to as aminosilicone or amodimethicones.

Silicone polymers are generally macromolecules with a molecular weight of at least 500 g/mol, preferably at least 1000 g/mol, more preferably at least 2500 g/mol and more preferably at least 5000 g/mol which comprise repeating organic units.

The maximum molecular weight of the silicone polymer depends on the degree of polymerization (number of polymerized monomers), and the batch size, and is also determined by the polymerization method. In the context of the present invention, it is preferable if the maximum molecular weight of the silicone polymers is not more than 107 g/mol, preferably not more than 10⁶ g/mol, and particularly preferably not more than 105 g/mol.

The silicone polymers comprise many Si—O repeat units, the Si atoms being able to bear organic residues such as, for example, alkyl groups or substituted alkyl groups. Alternatively, a silicone polymer is therefore also referred to as polydimethylsiloxane.

Corresponding to the high molecular weight of the silicone polymers, these are based on more than 10 Si—O repeat units, preferably more than 50 Si—O repeat units and particularly preferably more than 100 Si—O repeat units, very particularly preferably more than 500 Si—O units.

An amino-functionalized silicone polymer is understood to mean a functionalized silicone which bears at least one structural unit with an amino group. The amino-functionalized silicone polymer preferably bears a plurality of structural units with at least one amino group in each instance. An amino group is understood to mean a primary amino group, a secondary amino group and a tertiary amino group. All these amino groups can be protonated in an acidic environment and are then present in their cationic form.

In principle, it was possible to achieve positive effects of amino-functionalized silicone polymers (a1) when they bore at least one primary, at least one secondary and/or at least one tertiary amino group. However, dyeings with the highest color intensities were observed when an amino-functionalized silicone polymer (a1) was used in an agent containing at least one secondary amino group.

In a very particularly preferred embodiment, an agent according to the invention is characterized in that
(a1) at least one amino-functionalized silicone polymer having at least one secondary amino group.

The secondary amino groups(s) can be at different positions in the amino-functionalized silicone polymer. A particularly good effect was found when an amino-functionalized silicone polymer (a1) was used that had at least one, preferably multiple, structural units of the formula (Si-amino).

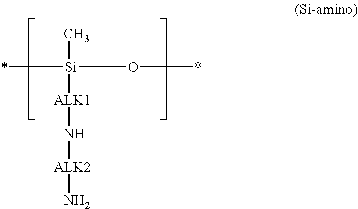

In the structural units of formula (Si-amino), the abbreviations ALK and ALK2 are each independently a linear or branched divalent C₁-C₂₀ alkylene group.

In an additional very particularly preferred embodiment, an agent according to the invention is characterized in that the agent contains at least one amino-functionalized silicone polymer (a1) which comprises at least one structural unit of formula (Si-amino),

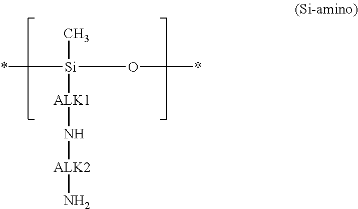

where
ALK1 and ALK2 represent, independently of one another, a linear or branched divalent C₁-C₂₀ alkylene group.

The position marked with an asterisk (*) always indicates the bond to other structural units of the silicone polymer. For example, the silicon atom adjacent to the asterisk can be bonded to an additional oxygen atom, and the oxygen atom adjacent to the asterisk can be bonded to an additional silicon atom or else to a C₁-C₆ alkyl group.

A divalent C₁-C₂₀ alkylene group can alternatively also be termed a double-bond C₁-C₂₀ alkylene group, which means that each moiety ALK1 or ALK2 can have two bonds.

In the case of ALK1, the silicon atom is bonded to the moiety ALK1, and the second bond is between ALK1 and the secondary amino group.

In the case of ALK2, the secondary amino group bonds with the moiety ALK2, and the second bond is formed between ALK2 and the primary amino group.

Examples of a linear divalent C₁-C₂₀ alkylene group are, for example, the methylene group (—CH₂—), the ethylene group (—CH₂—CH₂—), the propylene group (—CH₂—CH₂—CH₂—) and the butylene group (CH₂—CH₂—CH₂—CH₂—). The propylene group (—CH₂—CH₂—CH₂—) is particularly preferred. Starting at a chain length of 3 C atoms, divalent alkylene groups may also be branched. Examples of branched, divalent C₃-C₂₀ alkylene groups are (—CH₂—CH(CH₃)—) and (—CH₂—CH(CH₃)—CH₂—).

In an additional particularly preferred embodiment, the structural units of the formula (Si-amino) represent repeat units in the amino-functionalized silicone polymer (a1) so that the silicone polymer comprises multiple structural units of the formula (Si-amino).

In the following, particularly well-suited amino-functionalized silicone polymers (a1) with at least one secondary amino group are listed.

Dyeings with the greatest color intensities were obtained when an agent was applied on the keratin material which contains at least one amino-functionalized silicone polymer (a1) that comprises structural units of formula (Si-I) and formula (Si-II):

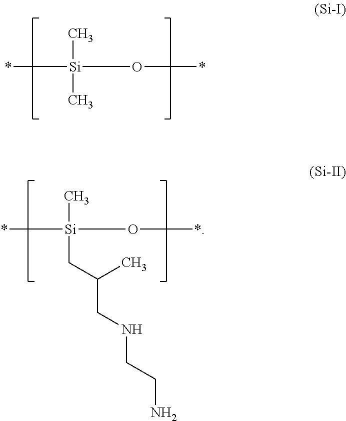

In an additional explicitly very particularly preferred embodiment, an agent according to the invention is characterized in that it contains at least one amino-functionalized silicone polymer (a1) which comprises structural units of formula (Si-I) and formula (Si-II):

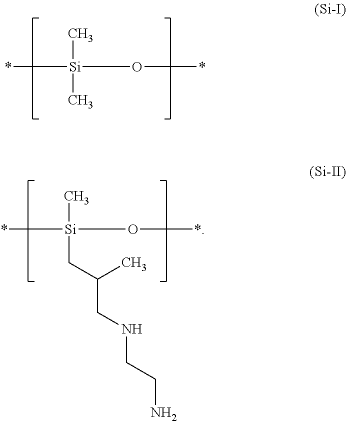

A corresponding amino-functionalized silicone polymer with the structural units (Si-I) and (Si-II) is, for example, the commercial product DC 2-8566 or Dowsil 2-8566 Amino Fluid which is sold commercially by Dow Chemical Company and which bears the designation “Siloxanes and Silicones, 3-[(2-Aminoethyl)amino]-2-methylpropyl Me, Di-Me-Siloxane” and the CAS number 106842-44-8. Another amino-functionalized silicone polymer with the structural units (Si-I) and (Si-II) is, for example, the commercial product DOWSIL™ AP-8568 Amino Fluid, which is likewise sold commercially by Dow Chemical Company.

In another preferred embodiment, an agent according to the invention is characterized in that it contains at least one amino-functional silicone polymer (a1) of the formula of the formula (Si-III):

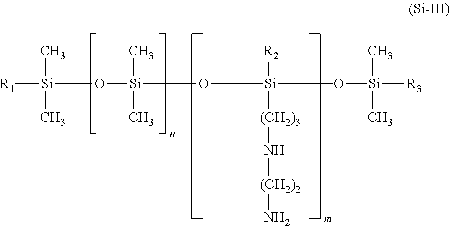

where
m and n denote numbers that are selected such that the sum (n+m) is in a range of 1 to 1000,
n is a number in a range of 0 to 999 and m is a number in a range of 1 to 1000,
R1, R2 and R3, which are identical or different, denote a hydroxyl group or a C₁-4 alkoxy group,
at least one of the R1 to R3 groups denoting a hydroxyl group.

Additional agents preferred according to the invention are characterized by their content of at least one amino-functional silicone polymer (a1) of the formula of the formula (Si-IV),

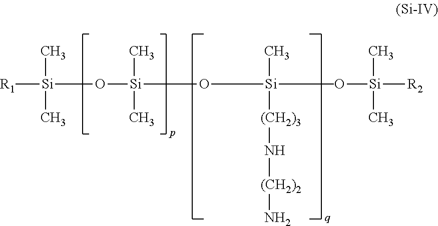

where
p and q denote numbers that are selected such that the sum (p+q) is in a range of 1 to 1000,
p is a number in a range of 0 to 999 and q is a number in a range of 1 to 1000, and
R1 and R2, which are different, denote a hydroxy group or a C₁-4 alkoxy group, at least one of the groups R1 to R2 denoting a hydroxy group.

The silicones of formula (Si-III) and (Si-IV) are different due to the moiety on the Si atom which bears the nitrogen-containing group. In formula (Si-III), R2 denotes a hydroxyl group or a C₁-4 alkoxy group while the residue in formula (Si-IV) is a methyl group. The individual Si moieties, which are labeled with the indices m and n or p and q, need not be present as blocks; instead, the individual units can also be distributed randomly, i.e., in the formulas (Si-III) and (Si-IV), each R1-Si(CH₃)₂ group is not necessarily bound to a —[O—Si(CH₃)₂] moiety.

Agents according to the invention which contain at least one amino-functional silicone polymer (a1) of the formula of the formula (Si-V) have also proven to be particularly effective with respect to the desired effects:

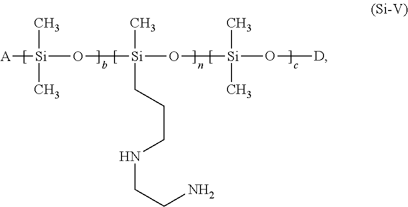

where
A represents an —OH, —O—Si(CH₃)₃, —O—Si(CH₃)₂OH, or —O—Si(CH₃)₂OCH₃ group,
D represents an —H, —Si(CH₃)₃, —Si(CH₃)₂OH, or —Si(CH₃)₂OCH₃ group, b, n and c stand for integers between 0 and 1000, with the proviso that
n>0 and b+c>0
at least one of the conditions A=—OH or D=—H is met.

In the aforementioned formula (Si-V), the individual siloxane units having the indices b, c, and n are randomly distributed, i.e., they are not necessarily block copolymers.

The agent (a) can also comprise one or more different amino-functionalized silicone polymers which are described by the formula (Si-VI):

M(RₐQ_{b}SiO_{(4-a-b)/2)x}(R_{c}SiO_{(4-c)/2)y}M (Si-VI).

In the above formula, R is a hydrocarbon or a hydrocarbon group having 1 to approximately 6 carbon atoms, Q is a polar group of general formula —R¹HZ, in which R¹ is a bivalent linking group bonded to hydrogen and the group Z, composed of carbon and hydrogen atoms, carbon, hydrogen, and oxygen atoms, or carbon, hydrogen and nitrogen atoms, and Z is an organic, aminofunctional group containing at least one aminofunctional group; “a” assumes values in a range of approximately 0 to approximately 2, “b” assumes values in a range of approximately 1 to approximately 3, “a”+“b” is less than or equal to 3, and “c” is a number in a range of approximately 1 to approximately 3, and x is a number in a range of 1 to approximately 2,000, preferably approximately 3 to approximately 50, and most preferably approximately 3 to approximately 25, and y is a number in a range of approximately 20 to approximately 10,000, preferably approximately 125 to approximately 10,000, and most preferably approximately 150 to approximately 1,000, and M is a suitable silicone end group as is known in the prior art, preferably trimethylsiloxy. Non-limiting examples of the groups represented by R include alkyl groups, such as methyl, ethyl, propyl, isopropyl, isopropyl, butyl, isobutyl, amyl, isoamyl, hexyl, isohexyl, and the like; alkenyl groups such as vinyl, halovinyl, alkylvinyl, allyl, haloallyl, and alkylallyl; cycloalkyl groups such as cyclobutyl, cyclopentyl, cyclohexyl, and the like; phenyl groups; benzyl groups; halohydrocarbon groups such as 3-chloropropyl, 4-bromobutyl, 3,3,3-trifluoropropyl, chlorocyclohexyl, bromophenyl, chlorophenyl, and the like; and sulfur-containing groups such as mercaptoethyl, mercaptopropyl, mercaptohexyl, mercaptophenyl, and the like; R is preferably an alkyl group containing 1 to approximately 6 carbon atoms, and most preferably R is methyl. Examples of R¹ include methylene, ethylene, propylene, hexamethylene, decamethylene, —CH₂CH(CH₃)CH₂—, phenylene, naphthylene, —CH₂CH₂SCH₂CH₂—, —CH₂CH₂OCH₂—, —OCH₂CH₂—, —OCH₂CH₂CH₂—, —CH₂CH(CH₃)C(O)OCH₂—, —(CH₂)₃C(O)OCH₂CH₂—, —C₆H₄C₆H₄—, —C₆H₄CH₂C₆H₄—; and —(CH₂)₃C(O)SCH₂CH₂—.

Z is an organic, amino-functional group containing at least one functional amino group. A possible formula for Z is NH(CH₂)_{z}NH₂, where z is 1 or more. Another possible formula for Z is —NH(CH₂)_{z}(CH₂)_{zz}NH, in which both z and zz are independently 1 or more, this structure comprising diamino ring structures, such as piperazinyl. Z is most preferably a —NHCH₂CH₂NH₂ group. Another possible formula for Z is N(CH₂)_{z}(CH₂)_{zz}NX₂ or —NX₂, where each X of X₂ is selected independently from the group consisting of hydrogen and alkyl groups having 1 to 12 carbon atoms, and zz is 0.

Q is most preferably a polar, amino-functional group of formula —CH₂CH₂CH₂NHCH₂CH₂NH₂. In the formulas, “a” assumes values in the range of about 0 to about 2, “b” assumes values in the range of about 2 to about 3, “a”+“b” is less than or equal to 3, and “c” is a number in the range of about 1 to about 3. The molar ratio of the RₐQ_{b} SiO_{(4-a-b>/2} units to the R_{c}SiO_{(4-c)/2} units is in the range of about 1:2 to 1:65, preferably from about 1:5 to about 1:65 and most preferably from about 1:15 to about 1:20. If one or more silicones of the above formula are used, then the various variable substituents in the above formula can be different in the various silicone components, present in the silicone mixture.

Within the scope of another preferred embodiment, an agent according to the invention is characterized in that it contains at least one amino-functional silicone polymer of the formula (Si-VII):

R′ₐG₃₋ₐ-Si(OSiG₂)ₙ-(OSiG_{b}R′_{2-b})ₘ—O—SIG₃₋ₐ-R′ₐ (Si-VII),

in which:
G is —H, a phenyl group, —OH, —O—CH₃, —CH₃, —O—CH₂CH₃, —CH₂CH₃, —O—CH₂CH₂CH₃, —CH₂CH₂CH₃, —O—CH(CH₃)₂, —CH(CH₃)₂, —O—CH₂CH₂CH₂CH₃, —CH₂CH₂CH₂CH₃, —O—CH₂CH(CH₃)₂, —CH₂CH(CH₃)₂, —O—CH(CH₃)CH₂CH₃, —CH(CH₃)CH₂CH₃, —O—C(CH₃)₃, or —C(CH₃)₃;
a represents a number between 0 and 3, in particular 0;
b represents a number between 0 and 1, in particular 1;
m and n are numbers whose sum (m+n) is between 1 and 2000, preferably between 50 and 150, n preferably assuming values of 0 to 1999 and in particular from 49 to 149, and m preferably assuming values of 1 to 2000, in particular from 1 to 10;
R′ is a monovalent group selected from:
   -Q-N(R″)—CH₂—CH₂—N(R″)₂
   -Q-N(R″)₂
   -Q-N⁺(R″)₃A⁻
   -Q-N⁺H(R″)₂ A⁻
   -Q-N⁺H₂(R″)A⁻
   -Q-N(R″)—CH₂—CH₂—N⁺R″H₂A⁻,
each Q representing a chemical bond, —CH₂—, —CH₂—CH₂—, —CH₂CH₂CH₂—, —C(CH₃)₂—, —CH₂CH₂CH₂CH₂—, —CH₂C(CH₃)₂—, or —CH(CH₃)CH₂CH₂—, R″ representing identical or different functional groups from the group —H, -phenyl, -benzyl, —CH₂—CH(CH₃)Ph, from the C₁₋₂₀ alkyl groups, preferably —CH₃, —CH₂CH₃, —CH₂CH₂CH₃, —CH(CH₃)₂, —CH₂CH₂CH₂H₃, —CH₂CH(CH₃)₂, —CH(CH₃)CH₂CH₃, —C(CH₃)₃, and A representing an anion preferably selected from chloride, bromide, iodide or methosulfate.

In another preferred embodiment, an agent according to the invention is characterized in that it contains at least one amino-functional silicone polymer (a1) of the formula (Si-VIIa):

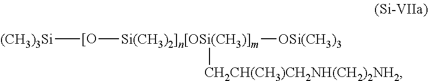

in which m and n are numbers of which the sum (m+n) is between 1 and 2000, preferably between 50 and 150, n preferably assuming values of 0 to 1999 and in particular from 49 to 149 and m preferably assuming values of 1 to 2000, in particular from 1 to 10.

These silicones are designated as trimethylsilylamodimethicones in accordance with the INCI Declaration.

In another preferred embodiment, an agent according to the invention is characterized in that it contains at least one amino-functional silicone polymer of the formula (Si-VIIb):

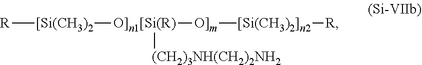

in which R denotes —OH, —O—CH₃, or a —CH₃ group and m, n1 and n2 are numbers whose sum (m+n1+n2) amounts to between 1 and 2000, preferably between 50 and 150, the sum (n1+n2) preferably assuming values from 0 to 1999 and in particular from 49 to 149 and m preferably assuming values from 1 to 2000, in particular from 1 to 10.

According to the INCI Declaration, these amino-functionalized silicone polymers are referred to as amodimethicones.

Irrespective of which amino-functional silicones are used, agents according to the invention are preferred that contain an amino-functional silicone polymer (a1) of which the amine value is above 0.25 meq/g, preferably above 0.3 meq/g, and in particular above 0.4 meq/g. The amine value here represents the milliequivalents of amine per gram of the amino-functional silicone. Said value can be determined by titration and may also be given in the unit mg KOH/g.

Furthermore, agents which contained a specific 4-morpholinomethyl-substituted silicone polymer (a1) are also suitable. This amino-functionalized silicone polymer comprises structural units of formulas (Si-VIII) and of the formula (Si-IX):

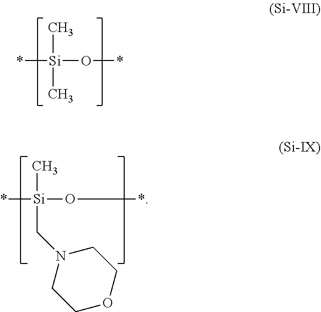

Corresponding 4-morpholinomethyl-substituted silicone polymers are described below.

A preferred amino-functionalized silicone polymer is known under the name amodimethicone/morpholinomethyl silsesquioxane copolymer and is commercially available in the form of the raw material Belsil ADM 8301 E from Wacker.

For example, a silicone which has structural units of formulas (Si-VIII), (Si-IX) and (Si-X) can be used as 4-morpholinomethyl-substituted silicone:

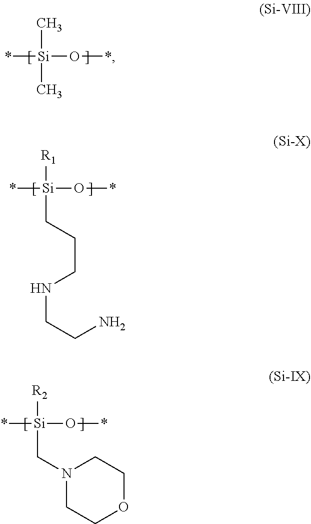

in which
R1 represents —CH₃, —OH, —OCH₃, —O—CH₂CH₃, —O—CH₂CH₂CH₃, or —O—CH(CH₃)₂;
R2 represents —CH₃, —OH, or —OCH₃.

Particularly preferred agents according to the invention contain at least one 4-morpholinomethyl-substituted silicone of formula (Si-XI):

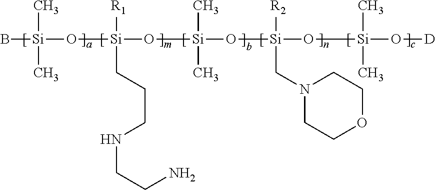

### (Si-XI)

where
   R1 represents —CH₃, —OH, —OCH₃, —O—CH₂CH₃, —O—CH₂CH₂CH₃, or —O—CH(CH₃)₂;
   R2 represents —CH₃, —OH, or —OCH₃.
   B represents an —OH, —O—Si(CH₃)₃, —O—Si(CH₃)₂OH, or —O—Si(CH₃)₂OCH₃ group,
   D represents an —H, —Si(CH₃)₃, —Si(CH₃)₂OH, or —Si(CH₃)₂OCH₃ group,
   a, b and c represent, independently of one another, integers between 0 and 1000, with the proviso that a+b+c>0
   m and n represent, independently of one another, integers between 1 and 1000,
   with the proviso that
      at least one of the conditions B=—OH or D=—H is met,
      the units a, b, c, m and n are distributed randomly or in blocks in the molecule.

Structural formula (Si-XI) is intended to indicate that the siloxane groups n and m do not necessarily have to be directly bonded to an end group B or D. Instead, in preferred formulas (Si-VI), a>0 or b>0 and, in particularly preferred formulas (Si-VI), a>0 and c>0; i.e., the terminal group B or D is preferably bonded to a dimethylsiloxy group. In formula (Si-VI) as well, the siloxane units a, b, c, m and n are preferably distributed randomly.

The silicones represented by formula (Si-VI) and used according to the invention can be trimethylsilyl-terminated (D or B=—Si(CH₃)₃), but they may also be dimethylsilylhydroxy-terminated at both ends or dimethylsilylhydroxy- and dimethylsilylmethoxy-terminated at one end. Within the context of the present invention, silicones which are particularly preferably used are selected from silicones in which:
B=—O—Si(CH₃)₂OH and D=—Si(CH₃)₃
B=—O—Si(CH₃)₂OH and D=—Si(CH₃)₂OH
B=—O—Si(CH₃)₂OH and D=—Si(CH₃)₂OCH₃
B=—O—Si(CH₃)₃ and D=—Si(CH₃)₂OH
B=—O—Si(CH₃)₂OCH₃ and D=—Si(CH₃)₂OH.
   These silicones lead to enormous improvements in the hair properties of hair treated with the agents according to the invention, and to greatly improved protection during oxidative treatment.

It has been found to be particularly advantageous if the agent according to the invention contains the amino-functionalized silicone polymers (a1) in certain quantity ranges. Particularly good results were obtained when the agent contains-based on the total weight of the agent-one or more amino-functionalized silicone polymers (a1) in a total amount of 0.1 to 8.0 wt. %, preferably 0.2 to 5.0 wt. %, more preferably 0.3 to 3.0 wt. %, and very particularly preferably 0.4 to 2.5 wt. %.

Within the scope of another particularly preferred embodiment, an agent according to the invention is characterized in that it contains-based on the total weight of the agent-one or more amino-functionalized silicone polymers (a1) in a total amount of 0.1 to 8.0 wt. %, preferably 0.2 to 5.0 wt. %, more preferably 0.3 to 3.0 wt. %, and very particularly preferably 0.4 to 2.5 wt. %.

### Platelet-Type Metallic Pigments (a2)

As the second essential component, the agent according to the invention contains at least one platelet-type metallic pigment (a2).

Pigments within the meaning of the present invention are understood to mean dyeing compounds which have a solubility of less than 0.5 g/L, preferably of less than 0.1 g/L, even more preferably of less than 0.05 g/L, at 25° C. in water. The method described below, for example, can be used to determine water solubility: 0.5 g of the pigment is weighed out in a beaker. A stir bar is added. Then one liter of distilled water is added. This mixture is heated to 25° C. while stirring with a magnetic stirrer for one hour. If still undissolved components of the pigment are visible in the mixture after this period, the solubility of the pigment is below 0.5 g/L. If the pigment-water mixture cannot be visually assessed due to the high intensity of the pigment that may be finely dispersed, the mixture is filtered. If a portion of undissolved pigments remains on the filter paper, the solubility of the pigment is below 0.5 g/L.

Metallic pigments are understood to mean pigments which comprise at least one metal and/or at least one metal alloy. Any metal suitable for metallic luster pigments is suitable as the metal. Such metals are, inter alia, iron and steel and, for example, platinum, zinc, chromium and molybdenum. Preferred metals are aluminum, copper, silver and gold. Aluminum is very particularly preferred. The metallic pigments according to the invention can also contain mixtures of metals which may be contained, for example, in the form of the corresponding alloys in the pigment. Suitable alloys are, for example, aluminum bronzes and brass.

Metals within the meaning of the present invention are the corresponding elements mentioned in the present case with the oxidation state 0, i.e., metal oxides such as aluminum oxide, iron oxide, zinc oxide or copper oxide are explicitly not to be understood as metals.

In a further very particularly preferred embodiment, an agent according to the invention is characterized in that it contains:
(a2) at least one platelet-type metallic pigment that comprises at least one metal from the group consisting of aluminum, copper, silver, gold, platinum, zinc, chromium, molybdenum and iron, particularly preferably aluminum.

In a further very particularly preferred embodiment, an agent according to the invention is characterized in that it contains:
(a2) at least one platelet-type metallic pigment comprising aluminum.

In a further explicitly very particularly preferred embodiment, an agent according to the invention is characterized in that it contains:
(a2) at least one platelet-type metallic pigment which is composed of, at least in part, a metal from the group consisting of aluminum, copper, silver, gold, platinum, zinc, chromium, molybdenum and iron, very particularly preferably aluminum.

In a further explicitly very particularly preferred embodiment, an agent according to the invention is characterized in that it contains:
(a2) at least one platelet-type metallic pigment which is composed of, at least in part, an elemental metal from the group consisting of aluminum, copper, silver, gold, platinum, zinc, chromium, molybdenum and iron, very particularly preferably aluminum.

The metallic pigments according to the invention are platelet-type. A platelet is understood to mean a three-dimensional body which can be regularly or irregularly shaped and whose width and depth have greater dimensions than its thickness. The average dimensions apply in each case as measurement points for the measurement of width, depth and thickness. Alternatively, the platelet-type constituents of the metallic pigment are also referred to as substrate plates.

Incident light is reflected as directed from a mirror onto the surfaces of the platelet shape of these metallic pigments. Its optical effect is based on the orientation of its platelets parallel to the surface of the surrounding system, which produces the metallic effect for an observer.

The platelet-type metallic pigment which is used in the dyeing agent according to the invention is present in the form of a large number of substrate plates. For example, a platelet-type aluminum pigment is a pigment consisting of a large number of substrate plates which comprise aluminum or are composed at least in part of elemental aluminum.

The platelet-type metallic pigments according to the invention preferably have a content of elemental metal of at least 50 wt. %, preferably of at least 60 wt. %, more preferably of at least 70 wt. %, even more preferably of at least 80 wt. %, even more preferably of at least 90 wt. %, in each case in relation to the weight of metallic pigment. If the platelet-type metallic pigment is coated, the aforementioned weight data refer to the weight of the uncoated metallic pigment. Within the meaning of the invention, the aforementioned content of elemental metal is also understood to mean the portion of the respective metal present in an alloy.

The substrate plates have a preferably monolithic structure. Monolithic in this context means consisting of a single self-contained unit without fractures, stratifications or inclusions, although structural changes may, however, occur within the substrate plates. The substrate plates are preferably composed homogeneously, i.e., there is no concentration gradient within the plates. In particular, the substrate plates are particularly preferably not composed of layers and do not have any particles distributed therein.

The size of the substrate plate can be matched to the respective application, in particular to the desired effect on the keratin material. The platelet-type metallic pigments used according to the present invention particularly preferably have a mean pigment diameter (D50) in a range from 0.5 μm (0.5 microns) to about 1 mm (1 millimeter), preferably from 3 μm (3 microns) to 500 μm (500 microns), even more preferably from 5 μm (5 microns) to 100 μm (100 microns) and very particularly preferably from 8 μm (8 microns) to 50 μm (50 microns).

In another particularly preferred embodiment, an agent according to the invention is characterized in that it contains:
(a2) at least one platelet-type metallic pigment having a mean pigment diameter (D50) of 0.5 μm to 1 mm, preferably of 3 μm to 500 μm, more preferably of 5 μm to 100 μm, and very particularly preferably of 8 μm to 50 μm.

The size distribution of the particles can be determined, for example, by means of laser granulometry. In this method, the particles can be measured in the form of a powder. The scattering of the irradiated laser light is detected in different spatial directions and evaluated according to the Fraunhofer diffraction theory. In this case, the particles are treated computationally as spheres. Thus, the determined diameters always refer to the equivalent spherical diameter determined over all spatial directions, regardless of the actual shape of the particles. The evaluation of the diffraction data is based on a model which targets the diameter of an equivalent sphere. Therefore, no absolute values are obtained, but the measured diameters have proven to be reliable relative values in the description of the size characteristic of platelet-type metallic pigments. The size distribution is determined which is calculated in the form of a volume mean in relation to the equivalent sphere diameter. This volume-averaged size distribution can be represented as a cumulative frequency distribution. For the sake of simplification, the cumulative frequency distribution is characterized by different characteristic values, for example the D50 value. The term “mean pigment diameter” or “D50” within the meaning of the present invention refers to the particle size, 50% of the aforementioned particle size distribution volume-averaged by means of laser granulometry being under the stated value and 50% of the aforementioned particle size distribution volume-averaged by means of laser granulometry being above the stated value. The measurements can be carried out, for example, using the HELOS particle size analyzer from Sympatec GmbH, Clausthal-Zellerfeld, Germany. Unless stated otherwise, the D50 value was determined with a Sympatec Helos-type device with Quixel wet dispersion. To prepare the sample, the sample to be investigated was pre-dispersed in isopropanol for a period of 3 minutes.

The mean thickness (h50) of the metallic pigments according to the invention is preferably in a range from 1 nm (1 nanometers) to about 500 nm (nanometers), preferably from about 1 nm (1 nanometers) to about 300 mm (1.5 mm), even more preferably from about 1 nm to 100 nm and very particularly preferably from 5 nm to 70 nm. The term “mean thickness” or “h50” within the meaning of the invention relates to the arithmetic mean of the thicknesses of at least 100 metallic pigments by means of scanning electron microscopy (SEM). The best possible orientation of the plates in the application medium is to be ensured here. For this purpose, the metallic pigments can be pretreated beforehand using suitable additives. Subsequently, the cured lacquer is abraded and, after conventional sample preparation, the transverse section is viewed in SEM. Only particles having a good orientation are selected for the counting. The mean thickness or the h50 value relates here to the uncoated metallic pigment.

The size-thickness ratio, also referred to as aspect ratio and expressed by the ratio of the mean size to the mean thickness, is preferably at least 80, preferably at least 200, more preferably at least 500 and particularly preferably more than 750.

In the course of the work leading to this invention, it was found that the use of metallic pigments from the group consisting of vacuum-metalized pigments (VMP) in the dyeing agents according to the invention leads to particularly good color results on the keratin material or the keratin fibers. Thus, it has been shown that the combination of VMP pigment (a2) and aminosilicone (a1) not only dyes the keratin material in a particularly long-lasting manner, but that these dyes also had a particularly high covering power and led to a very particularly uniform color result. Surprisingly, using the platelet-type pigments (a2), in particular using the VMP pigments and aminosilicone (a1), a dyed material with a particularly soft feel and pleasant hair feel was also obtained.

Vacuum-metalized pigments (VMP) are extremely thin metal plates which are produced in a specific method, so-called physical vapor deposition (PVD). These pigments have a uniform, smooth surface which does not flake and leads to an optical micro-mirror effect.

Vacuum-metalized pigments are characterized by extremely high gloss, an enormous covering power and unique optical properties. Due to their low thickness (approximately 5 to 70 nm) and their extremely smooth surfaces, they tend to cling very tightly to their substrate after their application. In the case of a very smooth substrate, this leads to an almost mirror-like appearance. Vacuum-metalized pigments (VMP) can be obtained, for example, by releasing metals or metal alloys of correspondingly coated films. Substrate plates which comprise a pigment metalized in a vacuum are also referred to as VMP substrate plates in the context of this application. VMP substrate plates of aluminum can be obtained, for example, by releasing aluminum from metalized films.

Vacuum-metalized substrate plates preferably have an average thickness (h50) of at most 70 nm, preferably less than 50 nm (nanometers), particularly preferably at most 35 nm (nanometers), particularly preferably at most 20 nm (nanometers). The average thickness of the substrate plates is at least 1 nm (nanometer), preferably at least 2.5 nm (nanometers), particularly preferably at least 5 nm (nanometers), for example at least 10 nm (nanometers). Preferred ranges for the thickness of the substrate plates are 2.5 to 70 nm, 5 to 50 nm, 10 to 35 nm, 2.5 to 30 nm and 5 to 25 nm. Preferably, each substrate plate has as uniform a thickness as possible. Due to the small thickness of the substrate plates, the pigment has a particularly high covering power.

Pigments based on metallic layers produced via PVD methods (physical vapor deposition) are described in more detail, for example, in U.S. Pat. No. 2,839,378 “Method of making metal flakes.” Described here is the production of mirror-like pigments with extremely thin layer thicknesses that are vapor-deposited on a substrate which is provided with a “release layer.” After the application of the metallic layers and detachment of the film, the pigments are comminuted to the desired particle size by means of mechanical stress. The process for producing metallic pigments by means of vapor deposition methods with a thickness of 35 to 45 nm is described in more detail in U.S. Pat. No. 4,321,087 and involves applying a release coat, the metalization process, the detachment process in a solvent bath, the concentration of the particles and the ultrasonic comminution to the desired pigment size.

In another particularly preferred embodiment, an agent according to the invention is characterized in that it contains:
(a2) at least one vacuum-metalized pigment as a platelet-type metallic pigment.

In another particularly preferred embodiment, an agent according to the invention is characterized in that it contains:
(a2) at least one vacuum-metalized pigment, which is at least partially composed of aluminum, as a platelet-type metallic pigment.

Dyeing with particularly good covering power and a good hair feel was obtained if uncoated metallic pigments were used as metallic pigments (a2).

In a further very particularly preferred embodiment, an agent according to the invention is characterized in that it contains:
(a2) at least one uncoated platelet-type metallic pigment.

In a further explicitly very particularly preferred embodiment, an agent according to the invention is characterized in that it contains:
(a2) at least one uncoated platelet-type aluminum pigment.

Vacuum-metalized pigments are commercially available from various suppliers. For example, the company Schlenk vakuum offers metalized pigments under the brand names Decometal® and Alegrace®.

| | Particle size | |
|---|---|---|
| Designation | (D50) | Morphology |
| Alegrace Marvelous A | 12 micrometers | VMP |
| 12/77-1 Bright Silver | | |
| Alegrace Marvelous A | 12 micrometers | VMP |
| 12/77-2 Platinum Silver | | |
| Alegrace Marvelous A | 12 micrometers | VMP |
| 12/77-3 White Silver | | |
| Alegrace Marvelous D | 12 micrometers | VMP |
| 12/77-1 Shiny Silver | | |

Further examples are Metalure® (produced by Avery Dennison, sold by ECKART), or Metasheen® (Ciba).

In the context of a further embodiment, the substrate plates may, however, also be passivated from metal or metal alloy, for example by anodizing (oxide layer) or chromating.

Uncoated VMP substrate plates, in particular those made of metal or metal alloy, reflect the incident light to a high degree and produce a light-dark flop, but no impression of color.

An impression of color can be produced, for example, from optical interference effects. Such pigments can be based on at least single-coated substrate plates. These manifest interference effects by superimposing differently refracted and reflected light beams.

Accordingly, pigments based on a VMP substrate plate which are coated can also be suitable. The substrate plate preferably has at least one coating B of a highly refractive metal oxide with a coating thickness of at least 50 nm. A coating A is preferably still between the coating B and the surface of the substrate plate. Optionally, another coating C, which is different from the underlying layer B, is on the layer B.

Suitable materials for coatings A, B and C are all substances that can be applied to the substrate plates in a film-like and permanent manner and, in the case of coatings A and B, have the required optical properties. In general, a coating of a part of the surface of the substrate plates is sufficient to obtain a pigment with a glossy effect. Thus, for example, only the upper and/or lower side of the substrate plates can be coated, the side face(s) being omitted. Preferably, the entire surface of the optionally passivated substrate plates, including the side surfaces, is covered by coating B. The substrate plates are therefore completely enveloped by coating B. This improves the optical properties of the pigment and increases the mechanical and chemical resilience the pigments. The above also applies to layer A and preferably also to layer C, if present.

Although a plurality of coatings A, B and/or C can always be present, the coated substrate plates preferably each have only one coating A, B and, if present, C.

The coating B is composed of at least one highly refractive metal oxide. Highly refractive materials have a refractive index of at least 1.9, preferably at least 2.0, and particularly preferably at least 2.4. The coating B preferably comprises at least 95 wt. %, particularly preferably at least 99 wt. %, of highly refractive metal oxide(s).

The coating B has a thickness of at least 50 nm. The thickness of coating B is preferably not more than 400 nm, particularly preferably at most 300 nm.

Highly refractive metal oxides suitable for coating B are preferably selectively light-absorbing (i.e., colored) metal oxides such as iron(III) oxide (α- and γ-Fe2O3, red), cobalt(II) oxide (blue), chromium(III) oxide (green), titanium(II) oxide (blue, usually in a mixture with titanium oxynitrides and titanium nitrides) and vanadium(V) oxide (orange) and mixtures thereof. Also suitable are colorless, highly-refractive oxides such as titanium dioxide and/or zirconium oxide.

Coating B can contain a selectively absorbing dye, preferably 0.001 to 5 wt. %, particularly preferably 0.01 to 1 wt. %, in each case based on the total amount of the coating B. Organic and inorganic dyes which can be stably incorporated into a metal oxide coating are suitable.

The coating A preferably has at least one low-refractive metal oxide and/or metal oxide hydrate. Preferably, coating A comprises at least 95 wt. %, particularly preferably at least 99 wt. %, low-refractive metal oxide (hydrate). Low-refractive materials have a refractive index of at most 1.8, preferably at most 1.6.

The low-refractive metal oxides suitable for coating A include, for example, silicon (di)oxide, silicon oxide hydrate, aluminum oxide, aluminum oxide hydrate, boric oxide, germanium oxide, manganese oxide, magnesium oxide and mixtures thereof, with silicon dioxide being preferred. The coating A preferably has a thickness from 1 to 100 nm, particularly preferably 5 to 50 nm, in particular preferably 5 to 20 nm.

The distance between the surface of the substrate plates and the inner surface of coating B is preferably at most 100 nm, particularly preferably at most 50 nm, in particular preferably at most 20 nm. Because the thickness of coating A and therefore the distance between the surface of the substrate plates and coating B is in the range indicated above, it can be ensured that the pigments have a high covering power.

If the pigment has only one layer A based on a VMP substrate plate, it is preferred that the pigment has a VMP substrate plate consisting of aluminum and a layer A of silicon dioxide. If the pigment has a layer A and a layer B based on a VMP substrate plate, it is preferred that the pigment has a VMP substrate plate consisting of aluminum, a layer A of silicon dioxide, and a layer B of iron oxide.

According to a further embodiment, the pigments have another coating C consisting of a metal oxide (hydrate) that is different from the underlying coating B. Suitable metal oxides are, for example, silicon (di)oxide, silicon oxide hydrate, aluminum oxide, aluminum oxide hydrate, zinc oxide, tin oxide, titanium dioxide, zirconium oxide, iron(III) oxide and chromium(III) oxide. Silicon dioxide is preferred.

The coating C preferably has a thickness of 10 to 500 nm, particularly preferably 50 to 300 nm. By providing the coating C, for example based on TiO₂, better interference can be achieved, while high covering power is maintained.

Layers A and C are in particular for corrosion protection as well as for chemical and physical stabilization. The layers A and C particularly preferably contain silicon dioxide or aluminum oxide which are applied by the sol gel method. This method comprises dispersing the uncoated VMP substrate plates, or the VMP substrate plates already coated with layer A and/or layer B, in a solution of a metal alkoxide such as tetraethyl orthosilicate or aluminum triisopropanolate (usually in a solution of organic solvent or a mixture of organic solvent and water with at least 50 wt. % organic solvent such as a C₁ to C₄ alcohol), and adding a weak base or acid for hydrolyzing the metal alkoxide, thereby forming a film of the metal oxide on the surface of the (coated) substrate plates.

Layer B can be produced, for example, by hydrolytic decomposition of one or more organic metal compounds and/or by precipitation of one or more dissolved metal salts and an optional subsequent post-treatment (for example, transferring formed hydroxide-containing layers into the oxide layers by tempering).

Although each of the coatings A, B and/or C can be composed of a mixture of two or more metal oxide (hydrates), each of the coatings is preferably composed of a metal oxide (hydrate).

The pigments based on coated VMP substrate plates preferably have a thickness from 70 to 500 nm, particularly preferably 100 to 400 nm, in particular preferably 150 to 320 nm, for example 180 to 290 nm. Due to the small thickness of the substrate plates, the pigment has a particularly high covering power. The small thickness of the coated substrate plates is achieved in particular because the thickness of the uncoated substrate plates is low, but also because the thicknesses of the coatings A and, if present, C are set to the smallest possible value. The thickness of coating B determines the color impression of the pigment.

The adhesion and abrasion resistance of pigments based on coated VMP substrate plates in the keratin material can be significantly increased by additionally modifying the outermost layer, depending on the structure of layer A, B or C, using organic compounds such as silanes, phosphoric acid esters, titanates, borates or carboxylic acids. The organic compounds are bonded to the surface of the outermost, preferably metal oxide-containing layer A, B or C. The outermost layer refers to the layer spatially furthest removed from the VMP substrate plate. The organic compounds are preferably functional silane compounds which can bind to the metal oxide-containing layer A, B or C. These may be either monofunctional or bifunctional compounds. Examples of bifunctional organic compounds include methacryloxypropenyltrimethoxysilane, 3-methacryloxypropyltrimethoxysilane, 3-acryloxypropyltrimethoxysilane, 2-acryloxyethyltrimethoxysilane, 3-methacryloxy-propyltriethoxysilane, 3-acryloxypropyltrimethoxysilane, 2-methacryloxyethyltriethoxysilane, 2-acryloxyethyltriethoxysilane, 3-methacryloxypropyltris(methoxyethoxy)silane, 3-methacryloxypropyltris(butoxyethoxy)silane, 3-methacryloxypropyltris(propoxy)silane, 3-methacryloxypropyltris(butoxy)silane, 3-acryloxy-propyltris(methoxyethoxy)silane, 3-acryloxypropyltris(butoxyethoxy)silane, 3-acryl-oxypropyltris(butoxy)silane, vinyltrimethoxysilane, vinyltriethoxysilane, vinylethyldichlorosilane, vinylmethyldiacetoxysilane, vinylmethyldichlorosilane, vinylmethyldiethoxysilane, vinyltriacetoxysilane, vinyltrichlorosilane, phenylvinyldiethoxysilane, or phenylallyldichlorosilane. Furthermore, a modification with a monofunctional silane, in particular an alkylsilane or arylsilane, can take place. This has only one functional group which can bind covalently to the surface of the pigment based on coated VMP substrate plates (i.e., to the outermost metal oxide-containing layer) or, when the covering is not complete, to the metal surface. The hydrocarbon functional group of the silane faces away from the pigment. Depending on the type and nature of the hydrocarbon functional group of the silane, a different degree of hydrophobicity of the pigment is achieved. Examples of such silanes are hexadecyltrimethoxysilane, propyltrimethoxysilane, etc. Particularly preferably, pigments based on silica-coated aluminum substrate plates are surface-modified with a monofunctional silane. Octyltrimethoxysilane, octyltriethoxysilane, hexadecyltrimethoxysilane and hexadecyltriethoxysilane are particularly preferred. As a result of the altered surface properties/hydrophobization, an improvement in terms of adhesion, abrasion resistance and orientation in the application can be achieved.

The amount of the platelet-type metallic pigments (a2) used can be selected depending on the desired color effect. Particularly good results were obtained when the agent contained-based on the total weight of the agent-one or more metallic pigments (a2) in a total amount of 0.01 to 10 wt. %, preferably 0.1 to 8 wt. %, more preferably 0.2 to 6 wt. %, and very particularly preferably 0.4 to 5.5 wt. %.

In another very particularly preferred embodiment, an agent according to the invention is characterized in that the agent contains-based on the total weight of the agent-one or more platelet-type metallic pigments (a2) in a total amount of 0.01 to 10 wt. %, preferably 0.1 to 8 wt. %, more preferably 0.2 to 6 wt. %, and very particularly preferably 0.4 to 5.5 wt. %. Further pigments (a3) different from (a2)

The dyeing agent according to the invention can additionally also contain one or more further pigments (a3) which are different from the platelet-type metallic pigments (a2). Suitable additional color pigments (a3) may be of inorganic and/or organic origin.

Preferred color pigments are selected from synthetic or natural inorganic pigments. Inorganic color pigments of natural origin can be produced, for example, from chalk, ocher, umbra, green soil, burnt Sienna or graphite. Furthermore, black pigments such as, for example, iron oxide black, chromatic pigments such as, for example, ultramarine or iron oxide red, and also fluorescent or phosphorescent pigments, can be used as inorganic color pigments.

Colored metal oxides, hydroxides and oxide hydrates, mixed phase pigments, sulfur-containing silicates, silicates, metal sulfides, complex metal cyanides, metal sulfates, chromates and/or molybdates are particularly suitable. Particularly preferred color pigments are black iron oxide (CI 77499), yellow iron oxide (CI 77492), red and brown iron oxide (CI 77491), manganese violet (CI 77742), ultramarine (sodium aluminum sulphosilicates, CI 77007, Pigment Blue 29), chromium oxide hydrate (CI77289), Iron Blue (ferric ferrocyanide, CI77510) and/or carmine (cochineal).

Color pigments which are likewise particularly preferred according to the invention are colored pearlescent pigments. These are usually based on mica and may be coated with one or more metal oxides. Mica is a phyllosilicate. The most important representatives of these silicates are muscovite, phlogopite, paragonite, biotite, lepidolite, and margarite. In order to produce the pearlescing pigments in conjunction with metal oxides, mica, primarily muscovite or phlogopite, is coated with a metal oxide.

As an alternative to natural mica, synthetic mica coated with one or more metal oxides(s) can also be used as a pearlescent pigment. Particularly preferred pearlescent pigments are based on natural or synthetic mica and are coated with one or more of the aforementioned metal oxides. The color of the respective pigments can be varied by varying the layer thickness of the metal oxide(s).

In another preferred embodiment, an agent according to the invention is characterized in that it additionally contains at least one inorganic pigment (a3), which is different from the platelet-type metallic pigment (a2) and is preferably selected from the group consisting of colored metal oxides, metal hydroxides, metal oxide hydrates, silicates, metal sulfides, complex metal cyanides, metal sulfates, and/or mica-based colored pigments which are coated with at least one metal oxide and/or a metal oxychloride.

Especially preferably, an agent for dyeing keratin material, in particular human hair, containing the following is therefore preferred:
(a1) at least one amino-functionalized silicone polymer, and
(a2) at least one platelet-type metallic pigment, and
(a3) at least one inorganic pigment which is different from the platelet-type metallic pigments (a2).

In another preferred embodiment, an agent according to the invention is characterized in that it contains at least one dyeing compound (a2) from the group consisting of pigments selected from mica-based pigments which are coated with one or more metal oxides from the group consisting of titanium dioxide (CI 77891), black iron oxide (CI 77499), yellow iron oxide (CI 77492), red and/or brown iron oxide (CI 77491, CI 77499), manganese violet (CI 77742), ultramarine (sodium aluminum sulfosilicates, CI 77007, Pigment Blue 29), chromium oxide hydrate (CI 77289), chromium oxide (CI 77288), and/or iron blue (ferric ferrocyanide, CI 77510).

Examples of particularly suitable color pigments are commercially available, for example, under the trade names Rona®, Colorona®, Xirona®, Dichrona® and Timiron® from the company Merck, Ariabel® and Unipure® from the company Sensient, Prestige® from the company Eckart Cosmetic Colors, and Sunshine® from the company Sunstar.

Very particularly preferred color pigments with the trade name Colorona® are, for example:
Colorona Copper, Merck, MICA, CI 77491 (IRON OXIDES)
Colorona Passion Orange, Merck, Mica, CI 77491 (IRON OXIDES), Alumina
Colorona Patina Silver, Merck, MICA, CI 77499 (IRON OXIDES), CI 77891 (TITANIUM DIOXIDE)
Colorona RY, Merck, CI 77891 (TITANIUM DIOXIDE), MICA, CI 75470 (CARMINE)
Colorona Oriental Beige, Merck, MICA, CI 77891 (TITANIUM DIOXIDE), CI 77491 (IRON OXIDES)
Colorona Dark Blue, Merck, MICA, TITANIUM DIOXIDE, FERRIC FERROCYANIDE
Colorona Chameleon, Merck, CI 77491 (IRON OXIDES), MICA
Colorona Aborigine Amber, Merck, MICA, CI 77499 (IRON OXIDES), CI 77891 (TITANIUM DIOXIDE)
Colorona Blackstar Blue, Merck, CI 77499 (IRON OXIDES), MICA
Colorona Patagonian Purple, Merck, MICA, CI 77491 (IRON OXIDES), CI 77891 (TITANIUM DIOXIDE), CI 77510 (FERRIC FERROCYANIDE)
Colorona Red Brown, Merck, MICA, CI 77491 (IRON OXIDES), CI 77891 (TITANIUM DIOXIDE)
Colorona Russet, Merck, CI 77491 (TITANIUM DIOXIDE), MICA, CI 77891 (IRON OXIDES)
Colorona Imperial Red, Merck, MICA, TITANIUM DIOXIDE (CI 77891), D&C RED NO. 30 (CI 73360)
Colorona Majestic Green, Merck, CI 77891 (TITANIUM DIOXIDE), MICA, CI 77288 (CHROMIUM OXIDE GREENS)
Colorona Light Blue, Merck, MICA, TITANIUM DIOXIDE (CI 77891), FERRIC FERROCYANIDE (CI 77510)
Colorona Red Gold, Merck, MICA, CI 77891 (TITANIUM DIOXIDE), CI 77491 (IRON OXIDES)
Colorona Gold Plus MP 25, Merck, MICA, TITANIUM DIOXIDE (CI 77891), IRON OXIDES (CI 77491)
Colorona Carmine Red, Merck, MICA, TITANIUM DIOXIDE, CARMINE
Colorona Blackstar Green, Merck, MICA, CI 77499 (IRON OXIDES)
Colorona Bordeaux, Merck, MICA, CI 77491 (IRON OXIDES)
Colorona Bronze, Merck, MICA, CI 77491 (IRON OXIDES)
Colorona Bronze Fine, Merck, MICA, CI 77491 (IRON OXIDES)
Colorona Fine Gold MP 20, Merck, MICA, CI 77891 (TITANIUM DIOXIDE), CI 77491 (IRON OXIDES)
Colorona Sienna Fine, Merck, CI 77491 (IRON OXIDES), MICA
Colorona Sienna, Merck, MICA, CI 77491 (IRON OXIDES)
Colorona Precious Gold, Merck, Mica, CI 77891 (Titanium dioxide), Silica, CI 77491 (IRON OXIDES), Tin oxide
Colorona Sun Gold Sparkle MP 29, Merck, MICA, TITANIUM DIOXIDE, IRON OXIDES, MICA, CI 77891, CI 77491 (EU)
Colorona Mica Black, Merck, CI 77499 (Iron oxides), Mica, CI 77891 (Titanium dioxide)
Colorona Bright Gold, Merck, Mica, CI 77891 (Titanium dioxide), CI 77491 (Iron oxides)
Colorona Blackstar Gold, Merck, MICA, CI 77499 (IRON OXIDES)

Additional particularly preferred color pigments with the trade name Xirona® are, for example:
Xirona Golden Sky, Merck, Silica, CI 77891 (Titanium Dioxide), Tin Oxide
Xirona Caribbean Blue, Merck, Mica, CI 77891 (Titanium Dioxide), Silica, Tin Oxide
Xirona Kiwi Rose, Merck, Silica, CI 77891 (Titanium Dioxide), Tin Oxide
Xirona Magic Mauve, Merck, Silica, CI 77891 (Titanium Dioxide), Tin Oxide.

In addition, particularly preferred color pigments with the trade name Unipure® are, for example:
Unipure Red LC 381 EM, Sensient CI 77491 (Iron Oxides), Silica
Unipure Black LC 989 EM, Sensient, CI 77499 (Iron Oxides), Silica
Unipure Yellow LC 182 EM, Sensient, CI 77492 (Iron Oxides), Silica

In another embodiment, the agent according to the invention can also additionally contain one or more dyeing compounds (a3) from the group of organic pigments.

The organic pigments according to the invention are correspondingly insoluble organic dyes or color lakes which may be selected, for example, from the group of nitroso, nitro, azo, xanthene, anthraquinone, isoindolinone, isoindoline, quinacridone, perinone, perylene, diketopyrrolopyorrole, indigo, thioindido, dioxazine, and/or triarylmethane compounds.

Particularly well suited organic pigments can for example include carmine, quinacridone, phthalocyanine, sorghum, blue pigments with the Color Index numbers CI 42090, CI 69800, CI 69825, CI 73000, CI 74100 or CI 74160, yellow pigments with the Color Index numbers CI 11680, CI 11710, CI 15985, CI 19140, CI 20040, CI 21100, CI 21108, CI 47000 or CI 47005, green pigments with the Color Index numbers CI 61565, CI 61570 or CI 74260, orange pigments with the Color Index numbers CI 11725, CI 15510, CI 45370 or CI 71105, and red pigments with the Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915 and/or CI 75470.

In another particularly preferred embodiment, an agent according to the invention is characterized in that it additionally contains at least one organic pigment (a3) preferably selected from the group consisting of carmine, quinacridone, phthalocyanine, sorghum, blue pigments with the Color Index numbers CI 42090, CI 69800, CI 69825, CI 73000, CI 74100 or CI 74160, yellow pigments with the Color Index numbers CI 11680, CI 11710, CI 15985, CI 9140, CI 20040, CI 21100, CI 21108, CI 47000 or CI 47005, green pigments with the Color Index numbers CI 61565, CI 61570 or CI 74260, orange pigments with the Color Index numbers CI 11725, CI 15510, CI 45370 or CI 71105, and red pigments with the Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915 and/or CI 75470.

Especially preferably, an agent for dyeing keratin material, in particular human hair, containing the following is therefore preferred:
(a1) at least one amino-functionalized silicone polymer, and
(a2) at least one platelet-type metallic pigment, and
(a3) at least one organic pigment.

The organic pigment can also be a color lake. The term color lake within the meaning of the invention is understood to mean particles which comprise a layer of absorbed dyes, with the unit consisting of particles and dye being insoluble under the above-mentioned conditions. The particles may be, for example, inorganic substrates which may be aluminum, silica, calcium borosilicate, calcium aluminum borosilicate or aluminum.

For example, the alizarin color lake can be used as the color lake.

Owing to their excellent light and temperature resistance, the use of the aforementioned pigments in the method according to the invention is very particularly preferred. It is further preferred if the pigments used have a certain particle size. It is therefore advantageous according to the invention if the at least one pigment has a mean particle size D₅₀ from 1.0 to 50 μm, preferably from 5.0 to 45 μm, preferably from 10 to 40 μm, in particular from 14 to 30 μm. The mean particle size D₅₀ can be determined, for example, using dynamic light scattering (DLS).

The pigments (a3) are preferably used in specific quantity ranges in the agent.

Particularly positive results were obtained when the agent contained-based on the total weight of the agent-one or more pigments (a3) in a total amount of 0.01 to 10.0 wt. %, preferably 0.1 to 5.0 wt. %, more preferably 0.2 to 2.5 wt. %, and very particularly preferably 0.25 to 1.5 wt. %.

In another very particularly preferred embodiment, an agent according to the invention is characterized in that the agent contains-based on the total weight of the agent-one or more pigments (a3) in a total amount of 0.01 to 10.0 wt. %, preferably 0.1 to 5.0 wt. %, more preferably 0.2 to 2.5 wt. %, and very particularly preferably 0.25 to 1.5 wt. %.

In another very particularly preferred embodiment, an agent according to the invention is characterized in that the agent contains-based on the total weight of the agent-one or more inorganic pigments (a3) in a total amount of 0.01 to 10.0 wt. %, preferably 0.1 to 5.0 wt. %, more preferably 0.2 to 2.5 wt. %, and very particularly preferably 0.25 to 1.5 wt. %.

In another very particularly preferred embodiment, an agent according to the invention is characterized in that the agent contains-based on the total weight of the agent-one or more organic pigments (a3) in a total amount of 0.01 to 10.0 wt. %, preferably 0.1 to 5.0 wt. %, more preferably 0.2 to 2.5 wt. %, and very particularly preferably 0.25 to 1.5 wt. %.

The number of shades of the resulting color result can be increased by the optional use of the additional inorganic or organic pigments (a3) in the dyeing agent according to the invention. Surprisingly, along with the color variation, a sufficiently high smoothness or an improved hair feel was also obtained if the platelet-type metallic pigments (a2) and the further inorganic and/or organic pigments (a3) were used in certain weight ratios to one another. It has been found to be particularly advantageous if the weight ratio of the total amount of the platelet-type metallic pigments (a2) contained in the agent to the total amount of the additional pigments (a3) contained in the agent, i.e., the weight ratio (a)/(a3), is from 5.0 to 0.1, preferably from 2.5 to 0.2, more preferably from 1.5 to 0.4 and very particularly preferably from 1.0 to 0.5.

Accordingly, in a further embodiment, very particularly preferred is an agent containing
(a1) at least one amino-functionalized silicone polymer, and
(a2) at least one platelet-type metallic pigment, and
(a3) at least one inorganic and/or organic pigment which is different from the platelet-type metallic pigments (a2),
the weight ratio of the total amount of the platelet-type metallic pigments (a2) contained in the agent to the total amount of the additional pigments (a3) contained in the agent, i.e., the weight ratio (a)/(a3), being from 5.0 to 0.1, preferably from 2.5 to 0.2, more preferably from 1.5 to 0.4 and very particularly preferably from 1.0 to 0.5.

In a further very particularly preferred embodiment, an agent according to the invention is characterized in that it contains:
(a2) at least one platelet-type metallic pigment and
(a3) at least one inorganic and/or organic pigment which is different from the platelet-type metallic pigments (a2),
the weight ratio of the total amount of the platelet-type metallic pigments (a2) contained in the agent to the total amount of the additional pigments (a3) contained in the agent, i.e., the weight ratio (a)/(a3), being from 5.0 to 0.1, preferably from 2.5 to 0.2, more preferably from 1.5 to 0.4 and very particularly preferably from 1.0 to 0.5.

### Water Content in the Agent

The agent described above is a ready-to-use agent which can be applied to the keratin material. This ready-to-use agent preferably possesses a low to medium water content. It has been found that particularly those agents are well-suited which contain—based on the total weight of the agent—0.1 to 70.0 wt. %, preferably 0.5 to 35.0 wt. %, more preferably 1.0 to 20.0 wt. %, and particularly preferably 1.5 to 7.5 wt. % water.

In another explicitly very particularly preferred embodiment, an agent according to the present invention is characterized in that it contains-based on the total weight of the agent-0 to 70.0 wt. %, preferably 0.1 to 35.0 wt. %, more preferably 0.2 to 20.0 wt. %, and particularly preferably 0.3 to 7.5 wt. % water.

### Solvent in the Agent

The use of solvents in the agent according to the invention has led to very good results. For this reason, the agent according to the invention can additionally contain at least one solvent as an optional component.

Suitable solvents that can be used are, for example, solvents from the group consisting of poly-C₁-C₆-alkylene glycols, 1,2-propylene glycol, 1,3-propylene glycol, 1,2-butylene glycol, dipropylene glycol, ethanol, isopropanol, diethylene glycol monoethyl ether, glycerol, phenoxyethanol, and benzyl alcohol. The use of polyethylene glycols is very particularly preferred.

In a further very particularly preferred embodiment, an agent according to the invention is characterized in that it contains at least one solvent from the group consisting of poly-C₁-C₆-alkylene glycols, 1,2-propylene glycol, 1,3-propylene glycol, 1,2-butylene glycol, dipropylene glycol, ethanol, isopropanol, diethylene glycol monoethyl ether, glycerol, phenoxyethanol, and benzyl alcohol, and very particularly preferably polyethylene glycols.

Alternatively, 1,2-propylene glycol is also referred to as 1,2-propanediol and bears the CAS numbers 57-55-6 [(RS)-1,2-dihydroxypropane], 4254-14-2 [(R)-1,2-dihydroxypropane], and 4254-153 [(S)-1,2-dihydroxypropane]. Ethylene glycol is alternatively also referred to as 1,2-ethanediol and bears the CAS number 107-21-1. Glycerol is alternatively also referred to as 1,2,3-propanetriol and bears the CAS number 56-81-5. Phenoxyethanol has the CAS number 122-99-6.

All the solvents described above are commercially available from various chemicals suppliers such as Aldrich or Fluka.

In the context of a further preferred embodiment, an agent according to the invention is characterized in that it contains-based on the total weight of the agent-one or more solvents from the group consisting of poly-C₁-C₆-alkylene glycols, 1,2-propylene glycol, 1,3-propylene glycol, 1,2-butylene glycol, dipropylene glycol, ethanol, isopropanol, diethylene glycol monoethyl ether, glycerol, phenoxyethanol, and benzyl alcohol, in a total amount of 10.0 to 99.0 wt. %, preferably 30.0 to 99.0 wt. %, more preferably 50.0 to 99.0 wt. %, and very particularly preferably 70.0 to 99.0 wt. %. Alkylene glycols of formula (AG)

A very particularly suitable solution is alkylene glycols of the formula (AG)

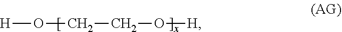

where
x denotes an integer from 1 to 10000, preferably an integer from 2 to 800, more preferably an integer from 3 to 600, even more preferably an integer from 3 to 400, and very particularly preferably an integer from 4 to 200.

In a further very particularly preferred embodiment, an agent according to the invention is therefore characterized in that it contains one or more alkylene glycols of the formula (AG),

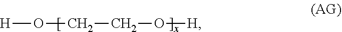

where
x denotes an integer from 1 to 10000, preferably an integer from 2 to 800, more preferably an integer from 3 to 600, even more preferably an integer from 3 to 400, and very particularly preferably an integer from 4 to 200.

The alkylene glycols of the formula (AG) are protic substances having at least one hydroxy group which, due to their repeating —CH₂—CH₂—O— unit, can also be referred to as polyalkylene glycols or polyethylene glycols insofar as x is a value of at least 2. In the alkylene glycols of formula (AG), x is an integer from 1 to 10,000. In the context of the work leading to this invention, it was found that these polyethylene glycols exhibit particularly favorable suitability for, on the one hand, improving the fastness properties of the dyeing agents and, on the other hand, also optimally adjusting the viscosity of the agents.

Depending on their chain length, polyethylene glycols are liquid or solid water-soluble polymers. Polyethylene glycols with a molecular mass between 200 g/mol and 400 g/mol are non-volatile liquids at room temperature. PEG 600 has a melting range of 17 to 22° C., and therefore a pasty consistency. With molecular masses above 3000 g/mol, the PEGs are solid substances and are commercially available as flakes or powders.

Especially the use of low molecular weight alkylene glycols (or, respectively, polyethylene glycols) has proven to be well-suited for achieving the object according to the invention. In the event of low molecular weight alkylene glycols (or polyethylene glycols) in the context of the present invention, x denotes an integer from 1 to 100, preferably an integer from 1 to 80, more preferably an integer from 2 to 60, even more preferably an integer from 3 to 40, even more preferably an integer from 4 to 20, and very particularly preferably an integer from 6 to 15.

In the context of another very particularly preferred embodiment, an agent according to the invention is characterized in that it contains at least one alkylene glycol of formula (AG-1),

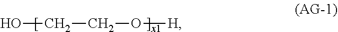

where
x1 is an integer from 1 to 100, preferably an integer from 1 to 80, more preferably an integer from 2 to 60, even more preferably an integer from 3 to 40, even more preferably an integer from 4 to 20, and very particularly preferably an integer from 6 to 15.

A very particularly preferred low molecular weight polyethylene glycol is PEG-8, for example. PEG-8 comprises, on average, 8 ethylene glycol units (x1=8), has an average molecular weight of 400 g/mol, and bears the CAS number 25322-68-3. PEG-8 is alternatively also referred to as PEG 400 and is commercially available, for example, from APS.

Additional well-suited low molecular weight polyethylene glycols are, for example, PEG-6, PEG-7, PEG-9 and PEG-10.

Another well-suited polyethylene glycol is PEG-32, for example. PEG-32 comprises 32 ethylene glycol units (x1=32), has a mean molar mass of 1500 g/mol and bears the CAS number 25322-68-3. PEG-32 is alternatively also referred to as PEG 1500 and can, for example, be purchased commercially from Clariant.

Furthermore, the use of high molecular weight polyethylene glycols for achieving the object according to the invention has also proven to be well suited.

High molecular weight polyethylene glycols within the meaning of the present invention can be represented by the formula (AG-2), the index number x2 standing for an integer from 101 to 10,000

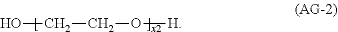

In the case of very well-suited high molecular weight polyethylene glycols, x2 denotes an integer from 101 to 1000, preferably an integer from 105 to 800, more preferably an integer from 107 to 600, even more preferably an integer from 109 to 400 and very particularly preferably an integer from 110 to 200.

In another very particularly preferred embodiment, an agent according to the invention is characterized in that it contains at least one alkylene glycol of formula (AG-2),

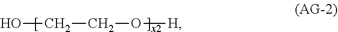

where
x2 denotes an integer from 101 to 1000, preferably an integer from 105 to 800, more preferably an integer from 107 to 600, even more preferably an integer from 109 to 400, and very particularly preferably an integer from 110 to 200.

A very particularly well-suited high molecular weight polyethylene glycol is, for example, PEG 6000, which can be obtained commercially from the National Starch company (China). The molecular weight of PEG 6000 is 6000 to 7500 g/mol, corresponding to an x2 value of 136 to 171.

Another well-suited polyethylene glycol is PEG 12000 which, for example, is commercially sold by CG chemicals under the trade name of polyethylene glycol 12000 S (or PEG 12000 S). The molecular weight of PEG 12000 is given at 10,500 to 15,000 g/mol, corresponding to an x2 value of 238 to 341.

Another well-suited polyethylene glycol is also PEG 20000 which is commercially available under the trade name polyglycol 20000 P or under the alternative name PEG-350 from Clariant. For PEG 20000, an average molecular weight of 20,000 g/mol is given which corresponds to an x2 value of 454.

Surprisingly, it has been found that dyeing agents which contain both a low molecular weight polyethylene glycol and a high molecular weight polyethylene glycol have particularly favorable application properties, since these agents have both very good fastness properties and are optimized with regard to their rheological profile.

Within the scope of another explicitly very particularly preferred embodiment, an agent according to the invention is characterized in that it contains:
at least one first alkylene glycol of formula (AG-1), where

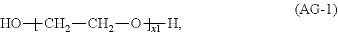

x1 denotes an integer from 1 to 100, preferably an integer from 1 to 80, more preferably an integer from 2 to 60, even more preferably an integer from 3 to 40, even more preferably an integer from 4 to 20, and very particularly preferably an integer from 6 to 15, and
at least one second alkylene glycol of formula (AG-2), where

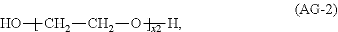

x2 denotes an integer from 101 to 1000, preferably an integer from 105 to 800, more preferably an integer from 107 to 600, even more preferably an integer from 109 to 400, and very particularly preferably an integer from 110 to 200.

To further optimize the application properties, the agent according to the invention comprises the alkylene glycol(s) (AG), preferably in certain quantity ranges which, for example—based on the total weight of the agent—can be in the range of 10.0 to 99.0 wt. %, preferably 30.0 to 99.0 wt. %, more preferably 50.0 to 99.0 wt. %, and very particularly preferably 70.0 to 99.0 wt. %.

Within the scope of another explicitly very particularly preferred embodiment, an agent according to the invention is therefore characterized in that it contains—based on the total weight of the agent—one or more alkylene glycols corresponding to formula (AG) in a total amount of 10.0 to 99.0 wt. %, preferably 30.0 to 99.0 wt. %, more preferably 50.0 to 99.0 wt. %, and very particularly preferably 70.0 to 99.0 wt. %.

The agent according to the invention preferably contains-based on the total weight of the agent—one or more alkylene glycols of formula (AG-1) in a total amount of 20.0 to 99.0 wt. %, preferably 40.0 to 95.0 wt. %, particularly preferably 60.0 to 90.0 wt. %.

The agent according to the invention preferably contains-based on the total weight of the agent—one or more alkylene glycols of formula (AG-2) in a total amount of 1.0 to 35.0 wt. %, preferably 3.0 to 30.0 wt. %, particularly preferably 4.0 to 25.0 wt. %.

Within the scope of another very particularly preferred embodiment, an agent according to the invention is characterized in that it contains-based on the total weight of the agent—one or more alkylene glycols of formula (AG-1) in a total amount of 20.0 to 99.0 wt. %, preferably 40.0 to 95.0 wt. %, more preferably 60.0 to 90.0 wt. %, and/or contains one or more alkylene glycols of formula (AG-2) in a total amount of 1.0 to 35.0 wt. %, preferably 3.0 to 30.0 wt. %, and particularly preferably 4.0 to 25.0 wt. %.

Within the scope of another very particularly preferred embodiment, an agent according to the invention is characterized in that it contains-based on the total weight of the agent—
one or more alkylene glycols of formula (AG-1) in a total amount of 20.0 to 99.0 wt. %, and
one or more alkylene glycols of formula (AG-2) in a total amount of 1.0 to 35.0 wt. %.

It is hereby understood that the sum of all of the alkylene glycols (AG), pigments (a2), (or the optionally additionally contained pigments (a3)) and amino-functionalized silicone polymers (a1) contained in the agents cannot be any more than 100 wt. %. If further, optional ingredients are also to be used in the agent, the total sum of said ingredients decreases to a corresponding extent to values less than 100 wt. %.

### Additional Ingredients in the Agent

The agents according to the invention may also contain even more active ingredients, auxiliaries, and additives, such as structurants like fatty components, glucose, maleic acid, and lactic acid; hair-conditioning compounds like phospholipids, for example lecithin and kephalins; perfume oils; dimethyl isosorbide and cyclodextrins; polymers such as anionic, nonionic, and cationic polymers; surfactants such as anionic, nonionic, cationic, zwitterionic, and amphoteric surfactants; fatty components; fiber structure-improving active ingredients, in particular mono-, di-, and oligosaccharides such as glucose, galactose, fructose, fruit sugar, and lactose; dyes for coloring the agent; anti-dandruff active ingredients such as piroctone olamine, zinc omadine, and climbazole; amino acids and oligopeptides; protein hydrolysates based on animals and/or plants, as well as in the form of their fatty acid condensation products or optionally anionically or cationically modified derivatives; vegetable oils; light stabilizers and UV blockers; active ingredients such as panthenol, pantothenic acid, pantolactone, allantoin, pyrrolidinonecarboxylic acids and their salts, and bisabolol; polyphenols, in particular hydroxycinnamic acids, 6,7-dihydroxycoumarins, hydroxybenzoic acids, catechins, tannins, leucoanthocyanidins, anthocyanidins, flavanones, flavones, and flavonols; ceramides or pseudoceramides; vitamins, provitamins, and vitamin precursors; plant extracts; fats and waxes such as fatty alcohols, beeswax, montan wax, and paraffins; swelling and penetrating agents such as glycerol, propylene glycol monoethyl ether, carbonates, hydrogen carbonates, guanidines, ureas, and primary, secondary, and tertiary phosphates; opacifiers such as latex, styrene/PVP, and styrene/acrylamide copolymers; pearlescent agents such as ethylene glycol mono- and distearate and PEG-3 distearate; and propellants such as propane-butane mixtures, N₂O, dimethyl ether, CO₂ and air.

The selection of these additional substances is made by the person skilled in the art according to the desired properties of the agents. With respect to other optional components and the employed amounts of said components, reference is made expressly to relevant manuals known to the person skilled in the art. The additional active ingredients and auxiliaries are used in the preparations according the invention preferably always in amounts of 0.0001 to 25 wt. %, in particular of 0.0005 to 15 wt. %, relative to the total weight of the particular agent.

### Methods for Dyeing Keratin Materials

The agents described above can be used outstandingly in methods for dyeing keratin material, in particular human hair.

A second object of the present invention is therefore a method for dyeing keratin material, in particular human hair, in which an agent as disclosed in detail in the description of the first object of the invention is applied to the keratin fibers and, if necessary, rinsed out again after an exposure time of 30 seconds to 45 minutes.

In other words, a second object of the invention is a method for dyeing keratin material, in particular human hair, comprising the following steps:
(1) applying a dyeing agent on the keratin material, the dyeing agent being an agent as has been disclosed in detail in the description of the first object of the invention,
(2) exposing the dyeing agent on the keratin material, and
(3) rinsing out the dyeing agent with water.

In step (1) of the method according to the invention, the agent of the first object of the invention is applied to the keratin material, which is very particularly preferably human hair.

In step (2) of the method according to the invention, the agent is then allowed to act on the keratin material after its application. In this context, various exposure times of, for example, 30 seconds to 60 minutes are conceivable.

However, a great advantage of the dyeing system according to the invention is that an intensive color result can be achieved even in very short periods after short exposure times. For this reason, it is advantageous for the application mixture to remain on the keratin material after application only for comparatively short periods of 30 seconds to 15 minutes, preferably 30 seconds to 10 minutes, and particularly preferably 1 to 5 minutes.

In another preferred embodiment, a method according to the invention is characterized by
(2) exposing the keratin material to the dyeing agent for a period of 30 seconds to 15 minutes, preferably 30 seconds to 10 minutes, and more preferably 1 to 5 minutes.

After the action of the application mixture on the keratin material, said keratin material is rinsed with water in step (3) of the process.

In one embodiment, the application mixture can be washed out with water only, i.e., without the aid of an after-treatment agent or a shampoo. The use of an after-treatment agent or conditioner in step (6) is also conceivable in principle.

To achieve the object according to the invention, and to increase the application comfort, however, it has been found to be very particularly preferred to rinse out the agent in step (3) exclusively with water, without the aid of an additional aftertreatment agent, shampoo, or conditioner.

In another preferred embodiment, a method according to the invention is characterized by
(3) rinsing out the dyeing agent exclusively with water.
   Method for Dyeing Keratin Material in which the Ready-to-Use Agent is First Produced.

As previously described, the agent of the first object of the invention is an agent ready for use which is either provided directly to the user in its ready-to-use form, or which is prepared by mixing various agents just prior to use.

In order to ensure a particularly fine distribution of the pigments according to the invention, it has been found to be very particularly preferred to produce the ready-to-use agent shortly before application by mixing at least two different agents.

In the context of a particularly preferred embodiment, the ready-to-use agent is accordingly prepared by mixing at least two different agents, the first of these two agents containing at least one amino-functionalized silicone polymer (a1) and the second agent containing at least one platelet-type metallic pigment (a2).

Another object of the present application is therefore a method for dyeing keratin material, in particular human hair, comprising the following steps:
(1) providing an agent (I), the agent (I) containing:
(a1) at least one amino-functionalized silicone polymer,
(2) providing an agent (II), the agent (II) containing:
(a2) at least one platelet-type metallic pigment,
(3) producing an application mixture by mixing the agents (1) and (II),
(4) applying the application mixture produced in step (3) on the keratin material,
(5) exposing the keratin material to the application mixture applied in step (4), and
(6) rinsing out the application mixture with water, the ingredients (a1) and (a2) having already been disclosed in detail in the description of the first object of the invention.

Agents (1) and/or (II) can optionally each contain one or more of the above-described additionally usable ingredients.

### Multi-Component Packaging Unit

To increase user comfort, the above-described agents in the form of a multicomponent packaging unit can be provided to the user.

Another object is therefore a multicomponent packaging unit (kit of parts) for dyeing keratin material, in particular human hair, comprising separately prepared:
a first container with an agent (1), the agent (1) containing:
(a1) at least one amino-functionalized silicone polymer, and
a second container with an agent (II), the agent (II) containing:
(a2) at least one platelet-type metallic pigment, the ingredients (a1) and (a2) having already been disclosed in detail in the description of the first object of the invention.

Another object of the present invention is therefore a multicomponent packaging unit (kit of parts) for dyeing keratin material, in particular human hair, comprising separately prepared:
a first container with an agent (1), the agent (1) containing:
(a1) at least one amino-functionalized silicone polymer,
a second container with an agent (II), the agent (II) containing:
(a2) at least one platelet-type metallic pigment, and
a third container with an agent (III); the agent (III) containing:
at least one alkylene glycol of the formula (AG), where

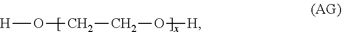

x denotes an integer from 1 to 10000, preferably an integer from 2 to 800, more preferably an integer from 3 to 600, even more preferably an integer from 3 to 400, and very particularly preferably an integer from 4 to 200,
the ingredients (a1) and (a2) and (AG) having already been disclosed in detail in the description of the first object of the invention.

Concerning the additional preferred embodiments of the methods according to the invention and the multicomponent packaging unit according to the invention, what has been said about the agent according to the invention applies mutatis mutantis.

### EXAMPLES

### 1. Formulations

The following ready-to-use dyeing agents were prepared (all data are in percent by weight unless stated otherwise):

| Ready-to-use dyeing agent | V1 | V2 | E1 |
|---|---|---|---|
| Colorona Precious Gold (Merck) | 0.5 | — | — |
| (Mica, CI 77891 (Titanium dioxide), | | | |
| Silica, CI 77491 (Iron oxides), tin | | | |
| oxide | | | |
| Colorona Bronze ((Merck), Mica, | — | 0.5 | — |
| iron oxide (CI 77019 and CI = 7791) | | | |
| VPM aluminum pigment (D(50) = | — | — | 0.5 |
| 12 microns), (Alegrace Marvelous D | | | |
| 12/77-1 Shiny Silver) | | | |
| Aminosilicone (Dow Corning 2-8566 | 0.74 | 0.74 | 0.74 |
| (siloxanes and silicones, 3-[(2- | | | |
| aminoethyl)amino]-2-methylpropyl | | | |
| Me, Di—Me—siloxane) | | | |
| PEG 6000 (polyethylene glycol, | 9.45 | 9.45 | 9.45 |
| molecular weight 6000 to 7500 | | | |
| g/mol) | | | |
| PEG 400 (polyethylene glycol, | up | up | up |
| molecular weight 400 g/mol) | to 100 | to 100 | to 100 |

### 2. Application

The ready-to-use agent produced beforehand was applied to hair strands (Kerling, type “Euronatur hair white” (ENH)) (bath ratio: 1 g agent per g of hair strand) and allowed to act for three minutes. Subsequently, the hair strands were washed thoroughly (1 minute) with water and dried.

### 3. Assessment of Wash Fastness and Hair Feel

The dyed strands were assessed by 5 trained persons with regard to the color intensity and the hair feel. Each strand was then washed manually. To this end, each strand was moistened with water, then a commercial shampoo (Schauma 7-Krauter) was applied to the strand (0.25 g shampoo per 1 g of hair) and massaged with the fingers 30 seconds. The strand was then rinsed for 1 minute under running lukewarm water, and the strand of hair was dried. The process described above corresponds to hair washing. For each additional hair wash, the process was repeated. After 3 hair washes, the strands were again compared by 5 trained persons with regard to the color intensity and the hair feel. A mean value was formed in each case from the individual values.

| | V1 | V2 | E1 |
|---|---|---|---|
| Color intensity directly after dyeing | +++ | +++ | +++ |
| Color intensity after 3 hair washes | ++ | ++ | +++ |
| Touch feel directly after dyeing | sticky, | sticky, | smooth, |
| | dull | dull | non-sticky |
| Touch feel after 3 hair washes | sticky | sticky | smooth, |
| | | | non-sticky |

+++ = very high color intensity
++ = medium color intensity
+ = low color intensity

### 3. Further Formulation Examples

The following ready-to-use dyeing agents were prepared (all data are in percent by weight unless stated otherwise):

| Ready-to-use dyeing agent | E2 | E3 | E4 |
|---|---|---|---|
| VPM aluminum pigment (D(50) = | 0.5 | 0.6 | 0.5 |
| 12 micrometers, Alegrace | | | |
| Marvelous D 12/77-1 Shiny Silver) | | | |
| Unipure Red LC 381 (CI 77491 | 0.8 | — | — |
| (iron oxides), silica) | | | |
| Unipure Red LC 3079 (Pigment | — | — | 0.4 |
| Red 7, CAS no. 5281-04-9) | | | |
| Goldmann Pigment Bue 60 | — | 0.95 | 0.4 |
| Plantacare 2000 UP (decyl | 0.5 | — | — |
| glucoside, water) | | | |
| Water (distilled) | 3.9 | — | — |
| PPG-14 butyl ether (UCON | 1.0 | 1.0 | 1.0 |
| FLUID AP) | | | |
| PPG-3 benzyl ether myristate | — | — | 3.3 |
| (Crodamol STS-LQ-(MH) | | | |
| Aminosilicone (Dow Corning 2- | 0.74 | 0.74 | 0.74 |
| 8566 (siloxanes and silicones, 3-[(2- | | | |
| aminoethyl)amino]-2-methylpropyl | | | |
| Me, Di—Me—siloxane) | | | |
| PEG 6000 (a12) (polyethylene | 19.4 | 9.4 | 14.0 |
| glycol, molecular weight 6000 to | | | |
| 7500 g/mol) | | | |
| PEG 400 (a11) (polyethylene | up | up | up |
| glycol, molecular weight 400 g/mol) | to 100 | to 100 | to 100 |

The hair strands dyed with the formulations E2, E3 and E4 had an intense coloring and a pleasant, soft feel.

1. An agent for dyeing keratin material, the agent comprising:
at least one amino-functionalized silicone polymer; and
at least one platelet-type metallic pigment.

2. The agent of claim 1, wherein the at least one amino-functionalized silicone polymer has at least one secondary amino group.

3. The agent of claim 1, wherein the at least one amino-functionalized silicone polymer comprises at least one structural unit of the formula (Si-amino): 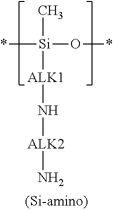
where
ALK1 and ALK2 represent, independently of one another, a linear or branched divalent C₁-C₂₀ alkylene group.

4. The agent of claim 1, wherein the at least one amino-functionalized silicone polymer comprises structural units of the formula (Si-I) and of the formula (Si-II):

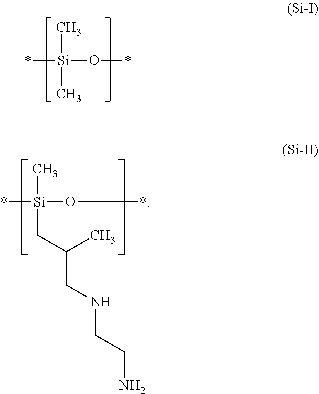

5. The agent of claim 1, wherein the at least one amino-functionalized silicone polymers is 0.1 to 8.0 wt. % of the total weight of the agent.

6. The agent of claim 1, wherein the at least one platelet-type metallic pigment comprises at least one metal from the group consisting of aluminum, copper, silver, gold, platinum, zinc, chromium, molybdenum, and iron.

7. The agent of claim 1, wherein the at least one platelet-type metallic pigment has a mean pigment diameter of 0.5 μm to 1 mm.

8. The agent of claim 1, wherein the at least one platelet-type metallic pigment comprises at least one vacuum-metalized pigment.

9. The agent of claim 1, wherein the at least one platelet-type metallic pigment comprises at least one uncoated platelet-type aluminum pigment.

10. The agent of claim 1, wherein the at least one platelet-type metallic pigment is 0.01 to 10 wt. % of the total weight of the agent.

11. The agent of claim 1, further comprising at least one inorganic pigment selected from the group consisting of colored metal oxides, metal hydroxides, metal oxide hydrates, silicates, metal sulfides, complex metal cyanides, metal sulfates, and mica-based colored pigments coated with at least one metal oxide or metal oxychloride.

12. The agent of claim 1, further comprising at least one organic pigment selected from the group consisting of carmine, quinacridone, phthalocyanine, sorghum, blue pigments with the Color Index numbers CI 42090, CI 69800, CI 69825, CI 73000, CI 74100, or CI 74160, yellow pigments with the Color Index numbers CI 11680, CI 11710, CI 15985, CI 19140, CI 20040, CI 21100, CI 21108, CI 47000, or CI 47005, green pigments with the Color Index numbers CI 61565, CI 61570, or CI 74260, orange pigments with the Color Index numbers CI 11725, CI 15510, CI 45370, or CI 71105, and red pigments with the Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915, or CI 75470.

13. The agent of claim 1, further comprising at least one inorganic pigment and/or organic pigment which is different from the at least one platelet-type metallic pigment,
wherein the weight ratio of the total amount of the at least one platelet-type metallic pigment to the total amount of the at least one inorganic pigment and/or organic pigment is 5.0 to 0.1.

14. The agent of claim 1, further comprising water, wherein the water is 0 to 70.0 wt. % of the total weight of the agent.

15. The agent of claim 1, further comprising at least one solvent from the group consisting of poly-C₁-C₆ alkylene glycols, 1,2-propylene glycol, 1,3-propylene glycol, 1,2-butylene glycol, dipropylene glycol, ethanol, isopropanol, diethylene glycol monoethyl ether, glycerol, phenoxyethanol, and benzyl alcohol.

16. The agent of claim 1, further comprising one or more polyalkylene glycols of the formula (AG), 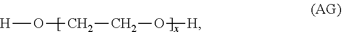
where
x denotes an integer from 1 to 10000,
wherein the one or more polyalkylene glycols of the formula (AG) are 10.0 to 99.0 wt. % of the total weight of the agent.

17. A method for dyeing keratin material, the method comprising: applying the agent of claim 1 to the keratin material for an exposure time of 30 seconds to 45 minutes.

18. The agent of claim 1, wherein the keratin material is human hair.

19. The agent of claim 6, wherein the at least one platelet-type metallic pigment comprises aluminum.

20. The method of claim 17, wherein the keratin material is human hair.
